(19) United States
(12) Patent Application Publication
(10) Pub. No.: US 2024/0216319 A1
(43) Pub. Date: Jul. 4, 2024

(54) TUMOR SUPPRESSANT AND PAIN RELIEF COMPOSITION FOR ORAL ADMINISTRATION

(71) Applicant: Hal Alper, Flowery Branch, GA (US)

(72) Inventor: Hal Alper, Flowery Branch, GA (US)

(21) Appl. No.: 18/457,928

(22) Filed: Aug. 29, 2023

Related U.S. Application Data
(60) Provisional application No. 63/403,178, filed on Sep. 1, 2022.

(51) Int. Cl. A61K 31/216; A61K 31/015; A61K 31/122; A61K 45/06; A61P 35/00

(52) U.S. Cl. CPC A61K 31/216; A61K 31/015; A61K 31/122; A61K 45/06; A61P 35/00

(57) ABSTRACT

Antitumor pharmaceutical compositions and methods of treatment for a variety of cancers, in which the antitumor compositions can be used alone or in combination with existing cancer therapies. The antitumor compositions can comprise or consist essentially of a combination of methyl jasmonate, cis-jasmone, and d-limonene. In a further aspect, the antitumor compositions can comprise or consist essentially of any two of methyl jasmonate, cis-jasmone, and d-limonene. Carriers or adjuvants such as squalene can be used with the compositions. This combination of components provides unexpected benefits, including synergistic enhancements cancer treatment and pain management when used with existing cancer therapies.

### CROSS-REFERENCE TO RELATED APPLICATIONS

None.

### TECHNICAL FIELD

This disclosure relates to compositions and methods for treating cancer and treating pain, including compositions that are useful to enhance the efficacy of current cancer treatments and chemotherapeutic agents.

### BACKGROUND

Chemical compounds derived from natural sources have found widespread applications in food, cosmetics, medicines, and countless other materials for human use. For example, compounds obtained from natural substances have been proposed as possible agents which may treat or help prevent cancer. Studies have examined the notion that consumption of certain constituents of plants, fruits, or vegetables or their components might afford some measure of cancer prevention or provide a therapeutic anticancer benefit. While promising, the ultimate utility of natural chemical compounds will depend upon their lack of toxicity towards healthy cells while attacking cancerous cells through any number of mechanisms.

Natural compounds which interfere with tumor development at any stage of promotion and progression by any mechanism are of potential clinical value. Therefore, there is a continuing search for natural chemical compounds which exhibit anticancer activity, but which lack toxicity against healthy cells. Desirable compounds include those which exhibit therapeutic activity themselves and those which can be used in conjunction with existing chemotherapeutic agents, without interfering with or hindering these chemotherapeutic agents.

### SUMMARY OF THE DISCLOSURE

This disclosure provides new chemical and pharmaceutical formulations for use in treating, halting the progress of, shrinking, or suppressing a wide variety of malignant and pre-malignant cancerous tumor cells in mammals, particularly humans. In an aspect, the new formulations can be used to for treating cancer in the absence of conventional chemotherapies or other cancer-treating drugs, or the new formulations can be used in combination with existing chemotherapies.

Many chemotherapeutic agents are highly toxic and their use may be accompanied by very unpleasant side-effect such as nausea, fatigue, digestive problems and the like, which can seriously reduce the quality of life for a cancer patient. Most chemotherapies must be administered over many months, and many lose their effectiveness after a certain time. Enhancing the effectiveness of chemotherapies is a desirable goal. Surprisingly, it has been discovered that the disclosed compositions can significantly enhance the effectiveness of existing cancer treatments when used in combination with current drugs including chemotherapies. These formulations can also be used in the absence of other anti-cancer drugs to reduce or shrink tumors and provide pain relief to a cancer patient.

The disclosed compositions include a combination of natural (non-synthetic) substances of plant origin which have all found use in other applications such as flavors and fragrances, rather than in the pharmaceutical industry, and which are safe for human consumption. One advantage of these compounds which are not controlled as drugs but which are recognized as safe is the ability to by-pass much of the usual and extensive testing necessary to show the safety and efficacy when used in pharmaceutical compositions. The components used in the present compositions have a long history of human exposure and even consumption without adverse effects. While these individual components can be used alone present compositions can be used alone, perhaps their most advantageous use is in conjunction with existing cancer therapies, including chemical or radiological.

While these new formulations use natural plant-derived compounds, some of which have been studied for possible antitumor activity, this disclosure explains the unexpectedly discovery of the synergistic anticancer effect which arises with a particular combination of plant-derived compounds. Also surprisingly, it has been found that a synergistic impact in efficacy, sometimes quite dramatic, can result when the disclosed formulations are used with other chemotherapeutic agents. That is, a remarkable shrinking of tumors can occur when these formulations are used as a combination therapy with current chemotherapies, and when these natural compounds are used in combination, rather than just one compound used with a chemotherapy. Perhaps most surprising was the reports of pain relief in cancer patients when the disclosed formulations are used in combination with chemotherapeutic agents.

One such compound is d-isomer of limonene, which is a cyclic monoterpene compound, and is the major component in citrus oils and citrus fruit peel. Limonene is a chiral molecule, and the main industrial source is citrus fruit peels from which a single enantiomer is obtained, the d-limonene isomer ((+)-limonene), which is the (R)-enantiomer. The d-isomer occurs more commonly in nature that the l-isomer, it has the fragrance of oranges, and d-limonene is a safe and useful flavoring agent in food manufacturing. Studies of d-limonene have been reported, for example, at: Cancer Res. 1992 (52) 4021-4026; J. Biol. Chem. 1995 (270) 17508-17512; and Phytother. Res. DOI: 10.1002/ptr.5165 (October 2014).

Plant-derived compounds such as the jasmonate family also have been studied in this regard. For example, jasmone is a volatile organic compound found in the oil of jasmine flowers and occurs naturally as the cis-isomer. Commercially, cis-jasmone has utility as a component of perfumes and cosmetic compositions. Methyl jasmonate is also produced in plants from jasmonic acid and is used by plants in plant defense and for flowering and fruit ripening. Both cis-jasmone and methyl jasmonate are safe to humans and occur in products used or consumed by humans. Studies of jasmonates have been reported, for example, at European Patent Specification EP 1 379 229 B1.

According to an aspect of the disclosure, the compositions for treating cancer can comprise or can consist essentially of any two or all three of methyl jasmonate, cis-jasmone, and d-limonene. In some embodiments, this combination of components can be used alone or in further combination with an adjuvant or adjunctive agent. For example, in embodiments, squalene can be used as an adjuvant with any combination of the methyl jasmonate, cis-jasmone, and d-limonene components in the formulation. Squalene can provide benefits as an adjunctive therapy with any and all cancers, and whether it exhibits anti-cancer properties itself in the combinations with the other components or more of an adjuvant effect, it has been found to be useful.

Squalene is a triterpene compound found naturally in shark liver oil, though it can be produced synthetically or derived from oil. Squalene is a common biological intermediate produced in plants and animals, and naturally produced squalene in the human body has a role in protecting and lubricating the skin. Among other uses, squalene is an important ingredient in some vaccine adjuvants, and its use in the present composition is as an optional adjuvant component.

Therefore, in an aspect, provided herein are compositions for treating cancer, the compositions comprising or consisting essentially of methyl jasmonate, cis-jasmone, and d-limonene. Another aspect of the disclosure provides for compositions for treating cancer, the compositions comprising or consisting essentially of any two of the compounds methyl jasmonate, cis-jasmone, and d-limonene. Still a further aspect provides a further combination of any two or all three of the compounds methyl jasmonate, cis-jasmone, and d-limonene, with squalene.

Another aspect of the disclosure provides that the individual components of the composition can be administered in a sequential manner, and the use of a composition comprising methyl jasmonate, cis-jasmone, d-limonene, and optionally squalene is not required. For example, it has been found that administering components individually when administered sublingually may permit more efficient absorption of the components into the system of the cancer patient. In this case, a rest period between administration of each individual components may be helpful.

In a further aspect, these disclosed compositions can be used alone or can be used in combination with chemotherapeutic agents to enhance existing cancer treatments. For example, the present compositions can be used in combination with any chemotherapeutic agent such as small molecules, biomolecules, or biologic chemotherapeutic agents. Each of methyl jasmonate (see Oser, B. L. and Ford, R. A. 1973; Recent progress in the consideration of flavoring ingredients under the Food Additives Amendment. 7. GRAS substances. Food Technol. 27(11): 56-57), cis-jasmone (21 C.F.R. § 172.515), and d-limonene (21 C.F.R. § 182.60) are considered safe for use in foods or cosmetics, and this underscores an advantage of the present compositions.

In a further aspect, when one, two, or all three of these components are plant-derived or plant-extracted and purified, rather than being synthesized and purified, the synergistic effect when used in combination with existing cancer treatments is also evident and in some cases even more striking. When these component compounds are plant-derived or plant-extracted and not synthetic and purified samples, and as with any plant-derived compound resulting from extraction and purification, a certain portion, for example from about 1-3 wt. % to about 5 wt. % (weight percent) of each compound sample can include closely related isomers, analogs, or homologs of the parent compound. In many cases, these related isomers, analogs, or homologs can have similar physiochemical properties. While not intending to be bound by theory, it is believed that the presence of these related isomers or close analogs contributes to the overall synergistic tumor killing effect, as compared to a more highly purified forms of the methyl jasmonate, cis-jasmone, and d-limonene compounds.

These three components methyl jasmonate, cis-jasmone, and d-limonene are liquid under conditions for their use and they may be present in any relative amounts. Therefore, the total concentration of plant-extracted methyl jasmonate, cis-jasmone, and d-limonene is 100 wt. % (weight percent) in the total composition when there are no additional components in the formulation and less than 100 wt. % when additional components such as a carrier or a chemotherapeutic agent or an adjuvant are present in the composition. Therefore, in another aspect the composition may further comprise a carrier, for example, a polar aprotic agent, a polar non-protic agent, or combinations thereof, or even a transdermal carrier for use in topical treatments. However, the methyl jasmonate, cis-jasmone, and d-limonene liquid composition is extremely effective for use in transdermal or sublingual administration without the need for transdermal carriers.

In an aspect, the formulations of this disclosure can be administered by injection, such as subcutaneously or intraperitoneally, or the formulations can be administered orally, sublingually, or topically. Sublingual absorption or oral administration are convenient and are effective. Direct injection into a tumor or any direct contact with a tumor can be used to administer the formulations. In another aspect, with suitable carrier components these formulations can be administered in the form of an ointment, a gel, or a cream. In some embodiments and with suitable carrier components, these formulations can be prepared for administration by inhalation or can be prepared for administration as a suppository.

It has been found that this formulation and the individual components of this formulation are nontoxic, target only cancer cells and not peripheral healthy cells, and work with existing chemotherapies to provide a synergistic effect shrink, suppress, or halt the growth of tumor cells. Use of the present formulation provides a multi-pronged approach to attack tumor cells in that the formulation targets multiple aspects of cancer cell genetics, functional morphology and biochemistry, and this multi-pronged approach can be expanded by using the disclosed formulation in combination with existing chemotherapies.

These and other aspects of the disclosure will be apparent to the skilled person when considered in combination with the detailed description and appended claims.

### BRIEF DESCRIPTION OF THE DRAWINGS

FIG. 1 illustrates the chemical structures of methyl jasmonate, cis-jasmone, d-limonene, and squalene.

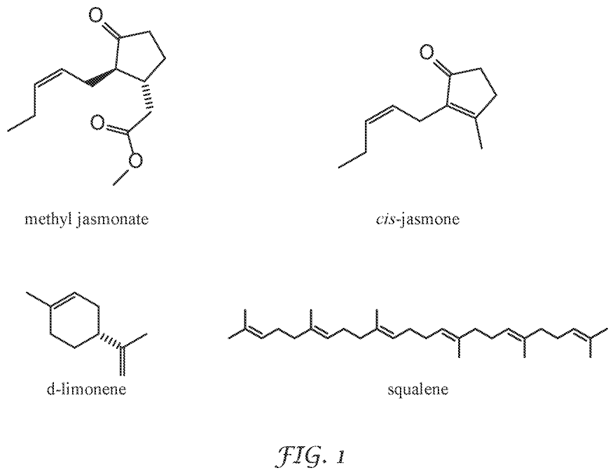

### DETAILED DESCRIPTION OF THE DISCLOSURE

### Definitions

To define more clearly the terms used herein, the following definitions are provided, and unless otherwise indicated or the context requires otherwise, these definitions are applicable throughout this disclosure. If a term is used in this disclosure but is not specifically defined herein, the definition from the IUPAC Compendium of Chemical Terminology, 2nd Ed (1997) can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

The terms “a,” “an,” and “the” are intended, unless specifically indicated otherwise, to include plural alternatives, for example, at least one. For instance, the disclosure of “a carrier” is meant to encompass one carrier or mixtures or combinations of more than one carrier unless otherwise specified.

The term “chemotherapeutic agent” and similar terms such as “chemotherapies” refers to a molecule or combination of molecules, large or small, useful to treat cancer. Non limiting examples of chemotherapeutic agents include organic compound traditionally referred to as small molecules, as well as biologic agents and other larger molecules used to treat cancer. Examples of chemotherapeutic agents include but are not limited to alkylating agents, anti-metabolites, anti-tumor antibiotics, plant alkaloids, microtubule inhibitors, DNA linking agents, biologics, bisphosphonates, hormones, other organic compounds, inorganic compounds, and other drugs known to be useful to treat cancer.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, by disclosing a weight percentage 70 wt. % to 80 wt. %, Applicant's intent is to recite individually 70 wt. %, 71 wt. %, 72 wt. %, 73 wt. %, 74 wt. %, 75 wt. %, 76 wt. %, 77 wt. %, 78 wt. %, 79 wt. %, and 80 wt. %, including any sub-ranges and combinations of sub-ranges encompassed therein, and these methods of describing such ranges are interchangeable. Accordingly, Applicant reserves the right to proviso out or exclude any individual members of any such group, including any sub-ranges or combinations of sub-ranges within the group, if for any reason Applicant chooses to claim less than the full measure of the disclosure.

Values or ranges may be expressed herein as “about”, from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed herein, and that each value is also herein disclosed as “about” that particular value in addition to the value itself. In aspects, “about” can be used to mean within 10% of the recited value, within 5% of the recited value, within 2% of the recited value, or within 1% of the recited value.

For the purposes of describing and defining the present teachings, the term “substantially” is utilized to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

All publications and patents mentioned in this disclosure are incorporated herein by reference for the purpose of describing and disclosing, for example, the constructs and methodologies that are described in the publications, which might be used in connection with the presently described invention or to more fully describe the state of the art to which the disclosed subject matter pertains. The publications discussed throughout the text are provided solely for their disclosure prior to the filing date of the present application. Nothing herein is to be construed as an admission that the inventors are not entitled to antedate such disclosure by virtue of prior invention. To the extent that any definition or usage provided by any document incorporated herein by reference conflicts with the definition or usage provided herein, the definition or usage provided herein controls.

These and other features, advantages and embodiments of the invention disclosed herein will be readily apparent to those exercising ordinary skill after reading the foregoing disclosures. Accordingly, while specific embodiments of the invention have been described in considerable detail, variations and modifications of those embodiments can be effected without departing from the spirit and scope of the invention as claimed.

### General Description

Disclosed herein are new compositions for pharmaceutical use in treating, halting the progress of, shrinking, or suppressing a wide variety of malignant and pre-malignant cancerous tumors in mammals, particularly humans. In an aspect, the new formulations include a combination of two or all three of the specific plant-derived compounds, namely methyl jasmonate, cis-jasmone, and d-limonene. Still a further aspect provides a further combination of any two or all three of the compounds methyl jasmonate, cis-jasmone, and d-limonene, with squalene. These compounds are miscible in each other and the resulting liquid composition can be used neat, containing no additional components other than methyl jasmonate, cis-jasmone, and d-limonene, in the disclosed treatment methods. Administration can be by any method, but most convenient administration is oral or sublingual, aided by the excellent sublingual absorption of the composition, or even topical administration.

Surprising results achieved with this specific combination of plant-derived compounds include the synergies of the combination over any single component of the formulation, synergies with existing chemotherapies, and the surprising report of pain relief by cancer patients. Further surprising results include the improved performance when even one, but preferably any two or all three of the plant-derived compounds in the composition are plant-derived or plant-extracted and purified, as compared to using the synthesized and purified components. This enhanced synergy when using the plant-derived or -extracted sources of any one or more of these components is particularly evident when used in combination with existing cancer treatments such as conventional chemotherapies. The best improvement in performance can occur when all three of the methyl jasmonate, cis-jasmone, and d-limonene are the result of extraction and purification of natural plant products. Further improvements in performance are obtained when squalene is used in any combination of methyl jasmonate, cis-jasmone, and d-limonene.

The compounds methyl jasmonate, cis-jasmone, and d-limonene are known to be plant hormones and/or products of secondary metabolism serving multiple functions. In this composition and method of treatment, the methyl jasmonate, cis-jasmone, and d-limonene are not merely nutritional supplements. Each of these components is non-toxic and has no history of adverse reactions with other drugs, and have been studied individually for their ability to suppress malignancies clinically (in vivo) or in in vitro lab experiments. There is also a long history of human use and consumption of these the components as common food and flavor additives and in perfumery.

The mechanism of action of these compounds as anti-cancer agents individually is not entirely understood, and therefore the mechanism of action of the combination of these compounds is also not understood. In one aspect, and while not intending to be bound by theory, predominate theories of action include the possible inhibition or expression of particular genes or interference with biochemical pathways such as the inhibition of function of the enzyme farnesyl transferase by d-limonene which can prevent attachment of sesquiterpenes to cancer cells. Attachment of a sesquiterpene to cancer cells can operate as a cloaking device against recognition of the cancer cell by the immune system. Both methyl jasmonate and cis-jasmone have the ability to kill mitochondria of cancer cells without adverse effect on healthy cells. Again, which not intending to be theory-bound, it is thought that this activity could be due to the higher metabolic rate of cancer cells causing them to build up the lethal concentration of these compounds faster. Alternatively, this activity may arise from some difference in the membrane permeability of these compounds between cancerous and non-cancerous cells. Both methyl jasmonate and cis-jasmone appear to permeate through cell membranes very efficiently. For example, one drop of a 40:40:20 mixture (either by volume or weight) of the methyl jasmonate, cis-jasmone, and d-limonene mixture topically to a subject's limb (for example, a knee) results in the subject reporting the taste of these compounds within several minutes, supporting the excellent permeability of the mixture and the compounds in the mixture.

### Pharmaceutical Compositions

According to an aspect of this disclosure, there is provided a pharmaceutical composition for treating cancer, the pharmaceutical composition comprising or consisting essentially of therapeutically effective amounts of methyl jasmonate, cis-jasmone, and d-limonene. This pharmaceutical composition may be referred to herein as simply a composition, but it can be used alone or in combination with conventional chemotherapies to treat cancer. In an aspect, one, two, or all three of the components of the methyl jasmonate, the cis-jasmone, and the d-limonene can be provided from plant extraction and purification.

A further aspect provides that the pharmaceutical composition for treating cancer can include the methyl jasmonate in a concentration of from 0.5 wt. % to 99 wt. % of the composition, the cis-jasmone in a concentration of from 0.5 wt. % to 99 wt. % of the composition, and the d-limonene in a concentration of from 0.5 wt. % to 99 wt. % of the composition, where these weight percentages are relative to the combined total methyl jasmonate, cis-jasmone, and d-limonene. The total weight percentage of the methyl jasmonate, the cis-jasmone, and the d-limonene in the composition can be 100 wt. % of the total composition, that is, the composition can consist essentially of methyl jasmonate, cis-jasmone, and d-limonene. The total weight percentage of the methyl jasmonate, the cis-jasmone, and the d-limonene in the composition also can be less than 100 wt. % of the total composition when the composition contains other components such as a pharmaceutically acceptable diluent or carrier or an adjuvant such as squalene. However, a carrier is not necessary as the neat liquid consisting essentially of these three components is very useful in topical, oral, or sublingual administration therapies.

Accordingly, when a weight percentage of any of the components methyl jasmonate, cis-jasmone, or d-limonene in the composition is recited, these weight percentages are reported either relative to the combined methyl jasmonate, cis-jasmone, and d-limonene only, or relative to the total composition which can include other components. Unless otherwise specified that the recited weight percentages are relative to the total or entire composition (that is, including adjuvants, carriers, and the like) or unless the context requires otherwise, the reported weight percentages are relative to the combined total of methyl jasmonate, cis-jasmone, and d-limonene only in the composition and do not include any carriers or adjuvants. For example, a composition that is described as comprising 30 wt. % methyl jasmonate, 30 wt. % cis-jasmone, and 40 wt. % d-limonene, these percentages are relative to the combination of these three components, regardless of whether this combination of three components is used alone or in further combination with a carrier or adjuvant or other formulation components. A composition that is described as consisting essentially of 30 wt. % methyl jasmonate, 30 wt. % cis-jasmone, and 40 wt. % d-limonene, these percentages are relative to the combination of these three components which is the same as the total composition. If a 30 wt. % methyl jasmonate, 30 wt. % cis-jasmone, and 40 wt. % d-limonene composition is relative to the combined methyl jasmonate, cis-jasmone, and d-limonene only, this composition can include further components. For example, if such a composition includes 30 g (grams) methyl jasmonate, 30 g cis-jasmone, and 40 g d-limonene, respectively, and this composition further includes 10 g squalene, the weight percentages of each of methyl jasmonate, cis-jasmone, and d-limonene in the total composition that includes squalene are now about 27.3 wt. %. 27.3 wt. %, and 36.4 wt. %, respectively, with 9.0 wt. % squalene also in the composition.

When a pharmaceutically acceptable carrier is used, it can comprise or be selected from a pharmaceutically acceptable alkyl alcohol, polysorbate, cyclodextrin, polyalkylene glycol, or primary amide, or any combination thereof. For example, the composition can further comprise a pharmaceutically acceptable carrier comprising or selected from ethanol, ethylene glycol, propylene glycol, butylene glycol, glycerin, TWEEN™ 20, TWEEN™ 40, TWEEN™ 80, hydroxypropyl-beta-cyclodextrin, polyethylene glycol, polypropylene glycol, polybutylene glycol, niacinamide, or any combination thereof.

When a pharmaceutically acceptable carrier, an adjuvant, or other additives are used, they can be used in any amounts that provide a composition suitable for administration by the intended method. In an aspect, for example, the pharmaceutically acceptable carrier, adjuvant, or other additive can be present in the composition in a concentration of from about 0.1 wt. % to about 30 wt. %, from about 0.2 wt. % to about 25 wt. %, from 0.3 wt. % to 20 wt. %, from 0.4 wt. % to 15 wt. %, or from 0.5 wt. % to 10 wt. %. In another aspect, the pharmaceutically acceptable carrier, adjuvant, or other additive can be present in the composition in a concentration of about 0.1 wt. %, 1 wt. %, 2 wt. %, 3 wt. %, 5 wt. %, 7 wt. %, 10 wt. %, 15 wt. %, 20 wt. %, 25 wt. %, 30 wt. %, 35 wt. %, or even higher. For example, when a pharmaceutically acceptable carrier is present in 15 wt. % in the composition, the 85 wt. % remainder of the composition is the combined total of any methyl jasmonate, cis-jasmone, and d-limonene present in the composition.

The pharmaceutical composition can include the methyl jasmonate, the cis-jasmone, and the d-limonene in a concentration selected independently from 0.5 wt. % to 99 wt. % of the composition, relative to the combined amounts of methyl jasmonate, cis-jasmone, and d-limonene. For example, the pharmaceutical composition for treating cancer can include the methyl jasmonate from 25 wt. % to 80 wt. % of the composition, the cis-jasmone from 25 wt. % to 80 wt. % of the composition, and the d-limonene from 1 wt. % to 50 wt. % of the composition, relative to the combined weights of these three components or relative to the weight of the total composition. In another aspect, the methyl jasmonate, the cis-jasmone, and the d-limonene can be present in the composition according to any of the weight percentages in the following Table 1, wherein the weight percentages are relative to the combined methyl jasmonate, cis-jasmone, and d-limonene.

TABLE 1
Exemplary concentrations of methyl jasmonate, cis-jasmone,
and d-limonene in the composition containing all three components
for treating cancer (wt. %), relative to the combined amounts
of methyl jasmonate, cis-jasmone, and d-limonene.

| | | methyl | |
|---|---|---|---|
| | d-limonene | jasmonate | cis-jasmone |
| Component | (wt. %) | (wt. %) | (wt. %) |
| Composition 1A | 0.5-99 | 0.5-99 | 0.5-99 |
| Composition 1B | 2-50 | 15-80 | 15-80 |
| Composition 1C | 2-45 | 15-70 | 15-70 |
| Composition 1D | 5-40 | 20-65 | 20-65 |
| Composition 1E | 5-40 | 20-60 | 20-60 |
| Composition 1F | 10-30 | 30-50 | 30-50 |
| Composition 1G | 15-25 | 35-45 | 35-45 |
| Composition 1H | 20 (or | 40 (or | 40 (or |
| | about 20) | about 40) | about 40) |

The exemplary relative concentrations of methyl jasmonate, cis-jasmone, and d-limonene in the Table 1 compositions also apply to the relative amounts of methyl jasmonate, cis-jasmone, and d-limonene when these components are administered sequentially, such as in a sublingual or oral administration method.

In a further aspect, the composition of the present disclosure can comprise a carrier, an adjuvant, or other components which can depending upon the administration method of the composition in from 0 wt. % to about 30 wt. %, and any of Compositions 1A through 1H from Table 1 present in 100 wt. % to about 70 wt. %. In other embodiments, the carrier, the adjuvant, or other components can be present from about 0.1 wt. % to about 30 wt. %, from about 0.2 wt. % to about 25 wt. %, from 0.3 wt. % to 20 wt. %, from 0.4 wt. % to 15 wt. %, or from 0.5 wt. % to 10 wt. %, with the balance in each case being the combined methyl jasmonate, cis-jasmone, and d-limonene, for example, any of Compositions 1A through 1H from Table 1.

According to a further aspect, this disclosure provides for compositions for treating cancer in which the compositions can comprise or can consist essentially of any two of the compounds methyl jasmonate, cis-jasmone, and d-limonene. Therefore compositions which comprise or consist essentially of methyl jasmonate and cis-jasmone, compositions which comprise of consist essentially of methyl jasmonate and d-limonene, and compositions which comprise of consist essentially of methyl jasmonate and cis-jasmone, are provided. When the compositions include any two of the compounds methyl jasmonate, cis-jasmone, and d-limonene, each compound can be present in any ratio. For example, each of the two compounds in the composition, methyl jasmonate, cis-jasmone, and d-limonene, can be present from 1-99 wt. % of the composition, wherein the weight percentages are relative to the combined any two of methyl jasmonate, cis-jasmone, and d-limonene.

Therefore, the total weight percentage of the two components in the composition can be 100 wt. % of the composition, that is, the composition for treating cancer can consist essentially of therapeutically effective amounts of any two of methyl jasmonate, cis-jasmone, and d-limonene. The total weight percentage of the two components in the composition also can be less than 100 wt. % of the composition, when the composition contains other components such as a pharmaceutically acceptable diluent or carrier. Any of the carriers and the amounts set out hereinabove can be used in the two-component compositions (containing any two of methyl jasmonate, cis-jasmone, and d-limonene), however as in the three-component composition, a carrier is not necessary as the neat liquid consisting essentially of any two of methyl jasmonate, cis-jasmone, and d-limonene can be readily used in topical, oral, or sublingual administration therapies. In another aspect, two of the components methyl jasmonate, cis-jasmone, and d-limonene can be present in the “two-component” compositions according to any of the weight percentages in the following Table 2.

TABLE 2
Exemplary concentrations of methyl jasmonate, the cis-jasmone, and
the d-limonene in compositions which contain any two of the three
compounds for treating cancer (wt. %), relative to the combination
of the any two of methyl jasmonate, cis-jasmone, and d-limonene.

| | | methyl | |
|---|---|---|---|
| | d-limonene | jasmonate | cis-jasmone |
| Component | (wt. %) | (wt. %) | (wt. %) |
| Composition 2A | 1-99 | 1-99 | 0 |
| Composition 2B | 1-99 | 0 | 1-99 |
| Composition 2C | 0 | 1-99 | 1-99 |
| Composition 2D | 20-80 | 20-80 | 0 |
| Composition 2E | 20-80 | 0 | 20-80 |
| Composition 2F | 0 | 20-80 | 20-80 |
| Composition 2G | 35-65 | 35-65 | 0 |
| Composition 2H | 35-65 | 0 | 35-65 |
| Composition 2I | 0 | 35-65 | 35-65 |
| Composition 2J | 50 (or | 50 (or | 0 |
| | about 50) | about 50) | |
| Composition 2K | 50 (or | 0 | 50 (or |
| | about 50) | | about 50) |
| Composition 2L | 0 | 50 (or | 50 (or |
| | | about 50) | about 50) |

The exemplary relative concentrations of methyl jasmonate, cis-jasmone, and d-limonene in the Table 2 compositions also apply to the relative amounts of any two of methyl jasmonate, cis-jasmone, and d-limonene when these components are administered sequentially, such as in a sublingual or oral administration method.

The composition of the present disclosure also can comprise a carrier, an adjuvant, or other components which can depending upon the administration method of the composition in from 0 wt. % to about 30 wt. %, and any of Compositions 2A through 2L from Table 2 present in 100 wt. % to about 70 wt. %. In other embodiments, the carrier, the adjuvant, or other components can be present from about 0.1 wt. % to about 30 wt. %, from about 0.2 wt. % to about 25 wt. %, from 0.3 wt. % to 20 wt. %, from 0.4 wt. % to 15 wt. %, or from 0.5 wt. % to 10 wt. %, with the balance in each case being the combination of two of methyl jasmonate, cis-jasmone, and d-limonene, for example, any of Compositions 2A through 2L from Table 2.

### Administration and Treatments

The pharmaceutical composition disclosed herein can provide a synergistic impact in chemotherapy efficacy, when the disclosed formulations are used in combination with other chemotherapeutic agents. Examples of chemotherapeutic agents that can be used in combination with the disclosed composition by co-administration include organic compounds traditionally referred to as small molecules, as well as biologic agents and other larger molecules used to treat cancer. Examples of chemotherapeutic agents include but are not limited to alkylating agents, anti-metabolites, anti-tumor antibiotics, plant alkaloids, microtubule inhibitors, DNA linking agents, biologics, bisphosphonates, hormones, other organic compounds, inorganic compounds, and other drugs known to be useful to treat cancer.

Specific examples of chemotherapeutic agents that can be used with the disclosed compositions by co-administration include, but are not limited to, aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, enhertu, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, or combinations thereof. Other chemotherapeutic agents that can be used in accordance with this disclosure include those set out in U.S. Pat. No. 10,495,645, which is incorporated herein by reference in its entirety.

The mode of administration of the composition described herein can comprise or can be selected from oral, transmucosal, sublingual, intranasal, insulfation, transdermal, intravenous, intra-arterial, intramuscular, intradermal, subcutaneous, injection, infusion, implantation, microinjection into a tumor, intraperitoneal, intraductal, intraventricular, intrathecal, intracranial, and the like.

In an aspect, the formulations of this disclosure can be administered by injection, such as subcutaneously or intraperitoneally, or can be administered orally or sublingually. Sublingual absorption or oral administration are most convenient and are effective. Direct injection into a tumor or any direct contact with a tumor can be used to administer the formulations.

According to another aspect, the particular cancer that can be treated can comprise or be selected from astrocytoma, bladder cancer, brain cancer, breast cancer, cervical cancer, colorectal cancer, endometrial cancer, gastric cancer, lung cancer, melanoma, leukemia, lymphoma, ovarian cancer, pancreatic cancer, prostate cancer, renal carcinoma, sarcoma, thyroid cancer, glioblastoma, multiple myeloma, myelodysplastic syndrome, mesothelioma, acute myeloid leukemia, childhood leukemia, chronic myeloid leukemia, myelodysplastic syndrome, Hodgkin's lymphoma, and Polycythemia Vera, as well as others.

Without being bound by theory, it is thought that the synergistic effect of the synergistic effect may be due to the ability of such combinations to access and attacks multiple sites via different cellular mechanisms at the same time, which may multiply the effectiveness. The surprising effect is that this effect is synergistic and not additive. That is, with each additional component of the methyl jasmonate, the cis-jasmone, the d-limonene, and the squalene being added to the composition, the resulting effect with respect to tumor suppression, cessation of tumor growth, and even pain relief is multiplied.

For example, one subject suffering from bladder cancer and on chemotherapy had recurring 3 cm to 5 cm (centimeter)-sized tumors with characteristic morphology removed every three months from the bladder, and the expectation was the bladder would eventually have to be removed. Following administration of d-limonene only, the subsequent surgical procedure revealed that the tumor size had reduced by about 10-foled, to millimeter-sized tumors, and the tumor morphology was ill-defined and amorphous, although still present. Upon administering the combination of methyl jasmonate in combination with the d-limonene, orally or subligually, the subsequent procedure revealed the complete absence of tumors, and this subject has been tumor free for three years.

As another example, the disclosed compositions have been used with a total of 10 subjects, who generally fall into two groups. The Group 1 subjects were in a very advanced stage of cancer and nearing the need for hospice care, having been told that their treatment options were essentially exhausted. In every case, within days of being administered the disclosed formulation, most often the 40 wt. % methyl jasmonate, 40 wt. % cis-jasmone, and 20 wt. % of d-limonene formulation, these subjects were able to leave hospice case and enjoy a reasonably good quality of life. These subjects for a large part reported being pain free for the first time in years. This ability to control and even eliminate pain is one of the most unexpected features of the disclosed formulation. The Group 2 subjects were those post-surgery with a high expectation of reoccurrence. In these subjects, not a single reoccurrence has been reported, even in cases where reoccurrence was expected.

There are substantial advantaged to the fact that these components are safe as well as effective. Methyl jasmonate has been a perfumery chemical for over a century, it is one of the components of jasmine flower scent, and it is a plant hormone produced when the plant is under attack. Cis-jasmone is also plant derived and safe and when used in combination with methyl jasmonate produces unexpected results over what would be expected for a mere additive effect. D-limonene targets a different part of the cancer cell, and again while not intending to be theory-bound, is thought to disrupt the cancer cell cloaking mechanism by possibly binding the enzyme farnesyl transferase (FT) which prevents sesquiterpene (ST) from attaching to the cancer cell and thereby preventing the immune system from recognizing the tumor cells. The use of d-limonene may be largely responsible for inhibiting tumor formation and it can likely alter tumor cell morphology. Squalene also has unique physiochemical properties and can provide benefits as an adjunctive therapy with any number of cancers.

Sublingual or oral administration of a liquid composition are the most convenient and easy to administer. Dosage can vary over a wide range with these compositions because they include components generally recognized as sage. The cancer patient can determine when some measure of pain relief and mobility and overall improvement is achieved. In an aspect, the dosage of the disclosed compositions can be higher in the early stages of administration and lower as the effect of the composition is manifested. In an aspect, the initial dosages for sublingual or oral administration can be, for example, from about 2 drops to about 20 drops of the composition, which can be administered for example one to three times a day for an initial period of time. This initial period of time can be, for example, from one to six weeks or more. In another aspect, this initial dose then may be decreased if desired to, for example, a range of from about 2 drops to about 10-20 drops of the composition, and this can be administered for example once daily for a second period of time, for example, for one to 4-6 weeks or more. In a further aspect, this second period dose then may be decreased if desired to, for example, 1-3 drops every day or every other day for a subsequent period of time, which can be for months or indefinitely to support and maintain the resulting effect. In these aspects, a single drop of the liquid composition can be considered about 0.05 mL (50 μL), which equals about 20 drops per milliliter (mL).

While this disclosure primarily describes the administration of a formulation containing all two, all three, or all four components disclosed herein, dosing regimens other than administering a mixture of all of the composition components can also provide good results. In the case of sublingual administration, the sequential treatment of each of the composition components individually, with a time period of a few minutes up to about an hour between administration of each component, has been used to good effect. For example, an sublingual treatment method can include: a first sublingual administration of two drops of methyl jasmonate followed by a first rest prior of about 10 minutes to an hour; a second sublingual administration of two drops of cis-jasmone followed by a second rest prior of about 10 minutes to an hour; and a third sublingual administration of one to two drops of d-limonene works well. If desired, following a rest period of about 10 minutes to an hour after the third sublingual administration of d-limonene, one or more drops of squalene can then be administered.

Further to this aspect of sequential administration, if desired, the order of administration of the components of the composition can be altered in any order, including administering two or more of the individual components at the same time. For example, a combination of 1-2 drops of methyl jasmonate and 1-2 drops of d-limonene can be administered sublingually, followed by a first rest period, which can be followed by sublingual administration of 1-2 drops of cis-jasmone. If squalene is used, the squalene can be administered at any time during the sequence, including before or after. Rest periods are generally from about 10 minutes to about an hour, for example, from about 15 minutes to about 45 minutes, or from about 10 minutes to about 30 minutes, and the like. While not intending to be bound by theory, it is thought that administering the composition components with some sequential aspect as described may benefit the absorption efficiency of the composition.

### EXAMPLES

In the following examples, the individuals involved were cancer patients who were under current treatment for their cancers by an oncologist, during the administration of the disclosed compositions. A composition containing 40 wt. % methyl jasmonate, 40 wt. % cis-jasmone, and 20 wt. % of d-limonene was used in the following Examples.

### Example 1. Patient 1

Female Patient 1 was diagnosed with stage 3 ovarian cancer in 2019, which was discovered to be platinum-resistant, recurrent epithelial ovarian cancer, which had spread to the colon. Chemotherapy treatments began in May-June, 2019 as an initial treatment to shrink the cancer and seek to avoid removal of a portion of the colon which would have required a colostomy. Platinum-based chemotherapy was effective, and the patient subsequently had surgery to remove the cancer that could be visualized which included removal of the appendix, lining of the stomach, and gallbladder. After six additional weeks of chemotherapy that followed, she “rang the bell” and was deemed to be cancer-free.

Two months later the cancer returned, but the patent went off of the chemotherapy for a period of six months for personal reasons. During this time the Cancer Antigen 125 (CA-125) levels went from 40 to 280 at the end of this time period. After this time, Patient 1 began a six-week stage 1 trial in November 2020, but the CA-125 levels had more than doubled from 280 to 600 over a five-week period. Suffering from severe pelvic pain, Patient 1 was admitted to the hospital, and her stomach was pumped. She was then diagnosed with a colon blockage from the cancer and underwent ileostomy surgery. The surgeon reported the presence of stage 4 tumors, the stage 1 trial was stopped, and on Dec. 28, 2020, Patient 1 was given 2 weeks to 2 months to live and recommended Hospice care begin.

Patient 1 started treatment with a composition containing 40 wt. % methyl jasmonate, 40 wt. % cis-jasmone, and 20 wt. % of d-limonene on Jan. 28, 2021. Administration was sublingual, using a dosage of two (2) drops of the composition twice a day for 3 to 4 weeks, followed by two (2) drops once a day for one month, followed by one (1) drop a day every other day. A CT scan of Patient 1 in April 2021 revealed that the cancer had not advanced. At this time, Patient 1 increased the dosage and began 10 to 12 drops per day under the tongue beginning on Apr. 12, 2021. During the administration of the composition containing methyl jasmonate, cis-jasmone, and d-limonene, Patent 1 reported significant improvement in her energy, mobility, appetite, and a reduction in pain both generally and in her bones.

### Example 2. Patient 2

Female Patient 2, 55 years old, was diagnosed with bladder cancer in July 2011 following a cystoscopy. A trans urethral resection was conducted on Aug. 25, 2011, and the pathology reported the presence of papillary urothelial lesion with ambiguous morphological features, locally comparable with urothelial papilloma/very low grade non-invasive urothelial carcinoma. Mild inflammation, focus of urothelial dysplasia, detrusor muscle present, and congruent controlled immunostains for Ck-20 and Ki-67 were reported. A tumor of approximately 5 cm in diameter was reported at posterior wall of the bladder.

Patient 2 returned every 3 months after the first cancer occurrence for cystoscopic evaluation. A second cancer recurrence was discovered on Jun. 17, 2013, which was again treated by resection. Patient 2 began chemotherapy treatment with Mitomycin on Jan. 2, 2014, and the subsequent chemotherapy on Jun. 5, 2014 involved a six-week course of BCG (Bacillus Calmette-Guerin solution) with Interferon treatment. Another cancer recurrence was diagnosed on Dec. 19, 2014, which required resection and Mitomycin chemotherapy for treatment.

A six-week course of BCG with Interferon began on Apr. 1, 2016, but tumors were again observed on Sep. 27, 2016, Jan. 10, 2017, and Aug. 15, 2017. A gemcitabine six-week course was started on Sep. 19, 2017, and tumors were again reported on Nov. 29, 2017, Mar. 13, 2018, and Jun. 14, 2018, requiring a BCG with Interferon treatment protocol which started Jul. 13, 2018 and again on Sep. 28, 2018.

Patient 2 began a course of treatment using a composition containing 40 wt. % methyl jasmonate, 40 wt. % cis-jasmone, and 20 wt. % of d-limonene on a daily basis in September 2018. One drop of this composition was taken by sublingual administration, and 1 drop was applied topically to the groin area daily. Patient 2 reported increased energy, stamina, and appetite after initiation of the treatment with the methyl jasmonate, cis-jasmone, and d-limonene composition. Cystoscopy evaluations were clear of tumors from Dec. 12, 2018 to May 2021. The most significant observation by Patient 2 was a reduction in joint and abdominal pain after being treated with this composition.

### Example 3. Patient 3

Adult female Patient 3, was diagnosed with a massive brain tumor on Jun. 15, 2020, which was surgically removed about two weeks later. Subsequent Pathology reports revealed the tumor to be a plasmacytoma, which is essentially a blood tumor which grows within soft tissue. After surgery, the Oncologist explained to Patient 3 that it was very likely that the patent either already had another tumor or that another tumor would develop quickly. Although scans did not show any other tumors at that time, in September and October of 2020, Patent 3 underwent radiation treatments to the area where the tumor had been.

After the initial diagnosis, the Oncologist indicated to Patent 3 that there was a 95% chance that he would develop another tumor within the next five years. Once a second tumor developed, Patient 3's cancer had progressed to multiple myeloma. At the end of 2020, Patient 3 began taking methyl jasmonate, cis-jasmone, and d-limonene in a sequential manner, approximately 1-2 drops each, with 10 minutes to 1 hour between compounds, each day. Subsequently, Patient 3 had scans every three months, and no additional tumors have developed. This continued to the 18-month mark, at which the Oncologist expressed his surprise that Patent 3 had not developed any further tumors. Patent 3 is approaching two years cancer-free, and again, the Oncologist continues to express his surprise that there have been no further cancer developments.

### Example 4. Sequential Treatment of Individual Composition Components

As disclosed, dosing regimens other than administering a mixture of all of the composition components can also provide good results. In the case of sublingual administration, the sequential treatment of each of the composition components individually, with a time period of a few minutes up to about an hour between administration of each component, has been used to good effect. For example, an sublingual treatment method can include: a first sublingual administration of two drops of methyl jasmonate followed by a first rest prior of about 15-30 minutes; a second sublingual administration of two drops of cis-jasmone followed by a second rest prior of about 15-30 minutes; and a third sublingual administration of one to two drops of d-limonene works well. If desired, following a rest period of 15-30 minutes after the third sublingual administration of d-limonene, one or more drops of squalene can then be administered. Also if desired, the order of administration of the components of the composition can be altered in any order, including administering two or more of the individual components at the same time.

### ASPECTS OF THE DISCLOSURE

The following aspects, features, and embodiments are set forth to further described and characterize the compositions and methods of this disclosure.

Aspect 1. A pharmaceutical composition for treating cancer, the pharmaceutical composition comprising or consisting essentially of methyl jasmonate, cis-jasmone, and d-limonene.

Aspect 2. A pharmaceutical composition for treating cancer, the pharmaceutical composition comprising or consisting essentially of methyl jasmonate, cis-jasmone, d-limonene and squalene.

Aspect 3. The pharmaceutical composition for treating cancer according to Aspect 2, wherein squalene is present in the total composition in a concentration of from about 0.1 wt. % to about 30 wt. %, and wherein the weight percentage of the methyl jasmonate, the cis-jasmone, and the d-limonene in the total composition is from 70 wt. % to 99.9 wt. %.

Aspect 4. The pharmaceutical composition for treating cancer according to any preceding Aspect, wherein the methyl jasmonate is present from 0.5 wt. % to 99 wt. % of the composition, the cis-jasmone is present from 0.5 wt. % to 99 wt. % of the composition, and the d-limonene is present from 0.5 wt. % to 99 wt. % of the composition, wherein the weight percentages are relative to the combined methyl jasmonate, cis-jasmone, and d-limonene.

Aspect 5. The pharmaceutical composition for treating cancer according to any preceding Aspect, wherein the methyl jasmonate is present from 25 wt. % to 80 wt. % of the composition, the cis-jasmone is present from 25 wt. % to 80 wt. % of the composition, and the d-limonene is present from 1 wt. % to 50 wt. % of the composition, wherein the weight percentages are relative to the combined methyl jasmonate, cis-jasmone, and d-limonene.

Aspect 6. The pharmaceutical composition for treating cancer according to any of Aspects 1 and 3-5, wherein the combined weight percentage of the methyl jasmonate, the cis-jasmone, and the d-limonene in the composition is 100 wt. % of the total composition.

Aspect 7. The pharmaceutical composition for treating cancer according to any of Aspects 1-5, wherein the combined weight percentage of the methyl jasmonate, the cis-jasmone, and the d-limonene in the composition is less than 100 wt. % of the total composition.

Aspect 8. The pharmaceutical composition for treating cancer according to any preceding Aspect, wherein the methyl jasmonate, the cis-jasmone, and the d-limonene are present in the composition according to any of the weight percentages in the following table, wherein the weight percentages are relative to the combined methyl jasmonate, cis-jasmone, and d-limonene:

| | | methyl | |
|---|---|---|---|
| | d-limonene | jasmonate | cis-jasmone |
| Component | (wt. %) | (wt. %) | (wt. %) |
| Composition 1A | 0.5-99 | 0.5-99 | 0.5-99 |
| Composition 1B | 2-50 | 15-80 | 15-80 |
| Composition 1C | 2-45 | 15-70 | 15-70 |
| Composition 1D | 5-40 | 20-65 | 20-65 |
| Composition 1E | 5-40 | 20-60 | 20-60 |
| Composition 1F | 10-30 | 30-50 | 30-50 |
| Composition 1G | 15-25 | 35-45 | 35-45 |
| Composition 1H | 20 (or | 40 (or | 40 (or |
| | about 20) | about 40) | about 40) |

Aspect 9. A pharmaceutical composition for treating cancer, the pharmaceutical composition comprising or consisting essentially of any two of methyl jasmonate, cis-jasmone, and d-limonene.

Aspect 10. A pharmaceutical composition for treating cancer, the pharmaceutical composition comprising or consisting essentially of: (a) any two of methyl jasmonate, cis-jasmone, and d-limonene; and (b) squalene.

Aspect 11. The pharmaceutical composition for treating cancer according to any of Aspects 9-10, wherein the pharmaceutical composition comprises or consists essentially methyl jasmonate and cis-jasmone.

Aspect 12. The pharmaceutical composition for treating cancer according to any of Aspects 9-10, wherein the pharmaceutical composition comprises or consists essentially of methyl jasmonate and d-limonene.

Aspect 13. The pharmaceutical composition for treating cancer according to any of Aspects 9-10, wherein the pharmaceutical composition comprises or consists essentially of cis-jasmone and d-limonene.

Aspect 14. The pharmaceutical composition for treating cancer according to any of Aspects 9-13, wherein each one of the any two of methyl jasmonate, cis-jasmone, and d-limonene are present from 1 wt. % to 99 wt. % of the composition, wherein the weight percentages are relative to the combined any two of methyl jasmonate, cis-jasmone, and d-limonene.

Aspect 15. The pharmaceutical composition for treating cancer according to any of Aspects 9 and 11-14, wherein the combined weight percentage of the any two of methyl jasmonate, cis-jasmone, and d-limonene is 100 wt. % of the total composition.

Aspect 16. The pharmaceutical composition for treating cancer according to any of Aspects 9-14, wherein the combined weight percentage of the any two of methyl jasmonate, cis-jasmone, and d-limonene is less than 100 wt. % of the total composition.

Aspect 17. The pharmaceutical composition for treating cancer according to any of Aspects 9-16, wherein the any two of methyl jasmonate, cis-jasmone, and d-limonene are present in the composition according to any of the weight percentages in the following table, wherein the weight percentages are relative to the combined any two of methyl jasmonate, cis-jasmone, and d-limonene:

| | | methyl | |
|---|---|---|---|
| | d-limonene | jasmonate | cis-jasmone |
| Component | (wt. %) | (wt. %) | (wt. %) |
| Composition 2A | 1-99 | 1-99 | 0 |
| Composition 2B | 1-99 | 0 | 1-99 |
| Composition 2C | 0 | 1-99 | 1-99 |
| Composition 2D | 20-80 | 20-80 | 0 |
| Composition 2E | 20-80 | 0 | 20-80 |
| Composition 2F | 0 | 20-80 | 20-80 |
| Composition 2G | 35-65 | 35-65 | 0 |
| Composition 2H | 35-65 | 0 | 35-65 |
| Composition 2I | 0 | 35-65 | 35-65 |
| Composition 2J | 50 (or | 50 (or | 0 |
| | about 50) | about 50) | |
| Composition 2K | 50 (or | 0 | 50 (or |
| | about 50) | | about 50) |
| Composition 2L | 0 | 50 (or | 50 (or |
| | | about 50) | about 50) |

Aspect 18. The pharmaceutical composition for treating cancer according to any of Aspects 1-17, wherein any one of the methyl jasmonate, the cis-jasmone, and the d-limonene is provided from plant extraction and purification.

Aspect 19. The pharmaceutical composition for treating cancer according to any of Aspects 1-17, wherein any two of the methyl jasmonate, the cis-jasmone, and the d-limonene are provided from plant extraction and purification.

Aspect 20. The pharmaceutical composition for treating cancer according to any of Aspects 1-8, wherein all three of the methyl jasmonate, the cis-jasmone, and the d-limonene are provided from plant extraction and purification.

Aspect 21. The pharmaceutical composition for treating cancer according to any of Aspects 1-5, 7-14, and 16-20, wherein the composition further comprises a pharmaceutically acceptable carrier.

Aspect 22. The pharmaceutical composition for treating cancer according to Aspect 21, wherein the pharmaceutically acceptable carrier comprises or is selected from a pharmaceutically acceptable alkyl alcohol, polysorbate, cyclodextrin, polyalkylene glycol, or primary amide, or any combination thereof.

Aspect 23. The pharmaceutical composition for treating cancer according to any of Aspects 21-22, wherein the pharmaceutically acceptable carrier comprises or is selected from ethanol, ethylene glycol, propylene glycol, butylene glycol, glycerin, TWEEN™ 20, TWEEN™ 40, TWEEN™ 80, hydroxypropyl-beta-cyclodextrin, polyethylene glycol, polypropylene glycol, polybutylene glycol, niacinamide, or any combination thereof.

Aspect 24. The pharmaceutical composition for treating cancer according to any of Aspects 21-23, wherein the pharmaceutically acceptable carrier is present in the pharmaceutical composition at a concentration of from about 0.1 wt. % to about 30 wt. %, from about 0.2 wt. % to about 25 wt. %, from 0.3 wt. % to 20 wt. %, from 0.4 wt. % to 15 wt. %, or from 0.5 wt. % to 10 wt. %.

Aspect 25. A method for treating cancer, the method comprising administering to a patient a therapeutically effective amount of the pharmaceutical composition according to any of Aspects 1-24.

Aspect 26. The method for treating cancer according to Aspect 25, wherein administering the pharmaceutical composition comprising or is selected from oral, transmucosal, sublingual, intranasal, insulfation, transdermal, intravenous, intra-arterial, intramuscular, intradermal, subcutaneous, injection, infusion, implantation, direct contact with a tumor, microinjection into a tumor, intraperitoneal, intraductal, intraventricular, intrathecal, or intracranial administration.

Aspect 27. The method for treating cancer according to any of Aspects 25-26, wherein the method further comprises co-administering a chemotherapeutic agent.

Aspect 28. The method for treating cancer according to Aspect 27, wherein the chemotherapeutic agent comprises, consists essentially of, or is selected from alkylating agents, anti-metabolites, anti-tumor antibiotics, plant alkaloids, microtubule inhibitors, DNA linking agents, biologics, bisphosphonates, hormones, or other compounds or drugs known to be useful to treat cancer.

Aspect 29. The method for treating cancer according to any of Aspects 27-28, wherein the chemotherapeutic agent comprises, consists essentially of, or selected from aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, or combinations thereof.

Aspect 30. The method for treating cancer according to any of Aspects 25-29, wherein the cancer comprises or is selected from astrocytoma, bladder cancer, brain cancer, breast cancer, cervical cancer, colorectal cancer, endometrial cancer, gastric cancer, lung cancer, melanoma, leukemia, lymphoma, ovarian cancer, pancreatic cancer, prostate cancer, renal carcinoma, sarcoma, thyroid cancer, glioblastoma, multiple myeloma, myelodysplastic syndrome, mesothelioma, acute myeloid leukemia, childhood leukemia, chronic myeloid leukemia, myelodysplastic syndrome, Hodgkin's lymphoma, or Polycythemia Vera.

Aspect 31. A method for treating pain associated with cancer, the method comprising administering to a patient a therapeutically effective amount of the pharmaceutical composition according to any of Aspects 1-24.

Aspect 32. A pharmaceutical composition for enhancing cancer treatment, the composition consisting essentially of methyl jasmonate, cis-jasmone, and d-limonene.

Aspect 33. A pharmaceutical composition for enhancing cancer treatment, the composition consisting essentially of methyl jasmonate, cis-jasmone, d-limonene, and squalene.

Aspect 34. A method for treating cancer, the method comprising administering to a patient therapeutically effective amounts of methyl jasmonate, cis-jasmone, and d-limonene individually in a sequential manner and in any order.

Aspect 35. The method for treating cancer according to Aspect 34, further comprising administering squalene in a sequential manner and in any order.

Aspect 36. A method for treating cancer, the method comprising administering to a patient therapeutically effective amounts of any two components selected from methyl jasmonate, cis-jasmone, and d-limonene separately from administration of the third non-selected component in a sequential manner and in any order.

Aspect 37. A method for treating cancer, the method comprising administering to a patient a therapeutically effective amounts of any two or any three components selected from methyl jasmonate, cis-jasmone, d-limonene, and squalene, separately from administration of the one or two non-selected components, respectively, in a sequential manner and in any order.

Aspect 38. A method for treating cancer according to Aspect 37, wherein the two non-selected components are administered together or in a sequential manner and in any order.

Aspect 39. The method for treating cancer according to any of Aspects 34-38, wherein the relative amount of methyl jasmonate is from 0.5 wt. % to 99 wt. %, the relative amount of cis-jasmone is from 0.5 wt. % to 99 wt. %, and the relative amount of d-limonene is from 0.5 wt. % to 99 wt. %, wherein the weight percentages are relative to the total methyl jasmonate, cis-jasmone, and d-limonene.

Aspect 40. A method for treating cancer according to any of Aspects 35 and 37-39, wherein the relative amount of squalene is from about 0.1 wt. % to about 30 wt. %, and the relative amount of the methyl jasmonate, the cis-jasmone, and the d-limonene combined is from 70 wt. % to 99.9 wt. %, wherein the weight percentages are relative to the total methyl jasmonate, cis-jasmone, d-limonene, and squalene.

What is claimed is:

1. A pharmaceutical composition for treating cancer, the pharmaceutical composition comprising methyl jasmonate, cis-jasmone, and d-limonene.

2. The pharmaceutical composition for treating cancer according to claim 1, wherein the composition further comprises squalene.

3. The pharmaceutical composition for treating cancer according to claim 2, wherein squalene is present in the composition in a concentration of from about 0.1 wt. % to about 30 wt. %, and wherein the weight percentage of the methyl jasmonate, the cis-jasmone, and the d-limonene in the composition is from 70 wt. % to 99.9 wt. %.

4. The pharmaceutical composition for treating cancer according to claim 1, wherein the methyl jasmonate is present from 25 wt. % to 80 wt. % of the composition, the cis-jasmone is present from 25 wt. % to 80 wt. % of the composition, and the d-limonene is present from 1 wt. % to 50 wt. % of the composition, wherein the weight percentages are relative to the combined methyl jasmonate, cis-jasmone, and d-limonene.

5. The pharmaceutical composition for treating cancer according to claim 1, wherein the combined weight percentage of the methyl jasmonate, the cis-jasmone, and the d-limonene in the composition is 100 wt. % of the total composition.

6. The pharmaceutical composition for treating cancer according to claim 1, wherein any one, any two, or all three of the methyl jasmonate, the cis-jasmone, and the d-limonene are provided from plant extraction and purification.

7. The pharmaceutical composition for treating cancer according to claim 1, wherein the composition further comprises a pharmaceutically acceptable carrier.

8. The pharmaceutical composition for treating cancer according to claim 7, wherein the pharmaceutically acceptable carrier comprises or is selected from a pharmaceutically acceptable alkyl alcohol, polysorbate, cyclodextrin, polyalkylene glycol, or primary amide, or any combination thereof.

9. The pharmaceutical composition for treating cancer according to claim 7, wherein the pharmaceutically acceptable carrier comprises or is selected from ethanol, ethylene glycol, propylene glycol, butylene glycol, glycerin, TWEEN™ 20, TWEEN™ 40, TWEEN™ 80, hydroxypropyl-beta-cyclodextrin, polyethylene glycol, polypropylene glycol, polybutylene glycol, niacinamide, or any combination thereof.

10. The pharmaceutical composition for treating cancer according to claim 7, wherein the pharmaceutically acceptable carrier is present in the pharmaceutical composition at a concentration of from about 0.1 wt. % to about 30 wt. %.

11. A method for treating cancer, the method comprising administering to a patient a therapeutically effective amount of the pharmaceutical composition according to claim 1.

12. The method for treating cancer according to claim 11, wherein administering the pharmaceutical composition is selected from oral, transmucosal, sublingual, intranasal, insulfation, transdermal, intravenous, intra-arterial, intramuscular, intradermal, subcutaneous, injection, infusion, implantation, direct contact with a tumor, microinjection into a tumor, intraperitoneal, intraductal, intraventricular, intrathecal, or intracranial administration.

13. The method for treating cancer according to claim 11, wherein the method further comprises co-administering a chemotherapeutic agent.

14. The method for treating cancer according to claim 13, wherein the chemotherapeutic agent is selected from alkylating agents, anti-metabolites, anti-tumor antibiotics, plant alkaloids, microtubule inhibitors, DNA linking agents, biologics, bisphosphonates, hormones, or other compounds or drugs known to be useful to treat cancer.

15. The method for treating cancer according to claim 13, wherein the chemotherapeutic agent is selected from aminoglutethimide, amsacrine, anastrozole, asparaginase, beg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, enhertu, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, vinorelbine, or combinations thereof.

16. The method for treating cancer according to claim 11, wherein the cancer comprises astrocytoma, bladder cancer, brain cancer, breast cancer, cervical cancer, colorectal cancer, endometrial cancer, gastric cancer, lung cancer, melanoma, leukemia, lymphoma, ovarian cancer, pancreatic cancer, prostate cancer, renal carcinoma, sarcoma, thyroid cancer, glioblastoma, multiple myeloma, myelodysplastic syndrome, mesothelioma, acute myeloid leukemia, childhood leukemia, chronic myeloid leukemia, myelodysplastic syndrome, Hodgkin's lymphoma, or Polycythemia Vera.

17. A pharmaceutical composition for treating cancer, the pharmaceutical composition comprising: (a) any two of methyl jasmonate, cis-jasmone, and d-limonene; and (b) squalene.

18. The pharmaceutical composition for treating cancer according to claim 17, wherein the pharmaceutical composition comprises methyl jasmonate and cis-jasmone.

19. The pharmaceutical composition for treating cancer according to claim 17, wherein the pharmaceutical composition comprises methyl jasmonate and d-limonene.

20. The pharmaceutical composition for treating cancer according to claim 17, wherein the pharmaceutical composition comprises cis-jasmone and d-limonene.

21. A method for treating cancer, the method comprising administering to a patient a therapeutically effective amount of the pharmaceutical composition according to claim 17.

22. A method for treating cancer, the method comprising administering to a patient therapeutically effective amounts of methyl jasmonate, cis-jasmone, and d-limonene individually in a sequential manner and in any order.

23. The method for treating cancer according to claim 22, further comprising administering squalene in a sequential manner and in any order.

24. A method for treating cancer, the method comprising administering to a patient therapeutically effective amounts of any two components selected from methyl jasmonate, cis-jasmone, and d-limonene separately from administration of the third non-selected component in a sequential manner and in any order.

25. A method for treating cancer, the method comprising administering to a patient a therapeutically effective amounts of any two or any three components selected from methyl jasmonate, cis-jasmone, d-limonene, and squalene, separately from administration of the one or two non-selected components, respectively, in a sequential manner and in any order.
